(19) United States
(12) Patent Application Publication
(10) Pub. No.: US 2025/0114474 A1
(43) Pub. Date: Apr. 10, 2025

(54) HEPARIN CONJUGATE AND METHOD FOR PREVENTING THROMBOGENESIS BY ADMINISTERING A PHARMACEUTICAL COMPOSITION COMPRISING THE SAME

(71) Applicant: National Yang Ming Chiao Tung University, Hsinchu (TW)

(72) Inventors: Chia-Ching CHANG, Hsinchu (TW); Chia-Yu CHANG, Hsinchu (TW); Chih-Yu YANG, Hsinchu (TW)

(21) Appl. No.: 18/907,862

(22) Filed: Oct. 7, 2024

Related U.S. Application Data
(60) Provisional application No. 63/543,117, filed on Oct. 9, 2023.

(51) Int. Cl. A61K 47/69; A61K 47/64; A61P 7/02

(52) U.S. Cl. CPC A61K 47/6901; A61K 47/64; A61P 7/02

(57) ABSTRACT

Provided are a composition and a method for preventing thrombogenesis. The composition includes a conjugate of heparin and a viral capsid protein, wherein the heparin is covalently bonded with the viral capsid protein.

### CROSS REFERENCE TO RELATED APPLICATION

This application claims the benefit of U.S. Provisional Application Ser. No. 63/543,117 filed on Oct. 9, 2023. The entirety of the application is incorporated herein by reference.

### BACKGROUND

### Technical Field

The present disclosure relates to a covalent conjugate of glycosaminoglycan, particularly heparin, its method of preparation, its pharmaceutical composition and therapeutic uses thereof.

### Description of Related Art

Heparins are a type of glycosaminoglycans (GAGs) composed of sulfated, linear, negatively-charged polysaccharides with carboxyl functional groups. They have a longstanding history of clinical utilization as blood anticoagulants in various medical procedures, including surgeries, cardiac catheterization, cardiopulmonary bypass, and hemodialysis. Functioning as an anticoagulant, heparin works by binding to antithrombin III and enhancing its inhibitory effects on thrombin (Factor IIa) and activated factor X (Factor Xa). Alongside its established role as an anticoagulant since the late 1930s, heparin is commonly prescribed to prevent conditions such as stroke, pulmonary embolism, and myocardial infarction. Moreover, heparin has demonstrated utility in preventing cancer-associated thrombotic diseases among cancer patients.

Despite being widely utilized, heparins employed in clinical settings have several inherent restrictions. Unfractionated heparin (UFH), sourced from animal tissues, possesses a molecular weight ranging from 3 to 30 KDa. The use of heparin, particularly UFH, is hindered by its short plasma half-life of 60 to 90 minutes, necessitating the administration of heparin via continuous infusion or intermittent subcutaneous injection, as heparin cannot be absorbed orally.

The limitations of heparin's pharmacokinetics can be attributed to its nonspecific interactions with proteins and cells. These include different plasma proteins (e.g., glycoproteins, lipoproteins, fibronectin, vitronectin, and fibrinogen), proteins secreted by platelets (e.g., platelet factor 4 (PF4) and high-molecular-weight von Willebrand factor (H-vWF)), and endothelial cells. As a consequence, the presence of heparin in patients' plasma is reduced, impacting its anticoagulant effectiveness. Therefore, the bioactivity of UFH is considered unpredictable and can lead to adverse effects such as heparin-induced thrombocytopenia (HIT). HIT occurs due to the non-specific binding of heparin to platelets and PF4, resulting in the production of antibodies against the PF4-heparin complex as part of an immune response to foreign proteins. These antibodies may bind to a neoepitope exposed on the PF4-heparin complex, triggering platelet activation and the release of procoagulant microparticles. This chain of events ultimately initiates thrombosis, followed by thrombocytopenia, which are characteristic signs of HIT. Severe complications arising from HIT can include arterial or venous thrombosis, affecting more than 20% of HIT patients, potentially resulting in limb loss or even death.

In order to address the challenges related to UFH, researchers developed low-molecular-weight heparin (LMWH), which offer improved bioavailability and reduced risks compared to UFH. Subsequently, in the early 2000s, ultralow molecular weight heparins (ULMWH) were introduced as even more advantageous alternatives to UFH. However, the convenience of ULMWH is counterbalanced by its higher cost and potential side effects. For instance, in the case of patients undergoing hemodialysis, UFH remains the preferred option due to its affordability, availability, and compatibility with the dialysis process.

Thus, there is still an unmet need for a type of heparin that exhibits an extended plasma half-life, improved bioavailability, and reduced non-specific binding, so as to ensure the development of a safer anticoagulant with enhanced pharmacokinetic properties.

### SUMMARY

In view of the foregoing, the present disclosure provides a conjugate of heparin and a viral capsid protein VP28. The conjugate of the present disclosure provides a non-immunogenic nanocomplex with an average hydrodynamic size that evades renal filtration and reticuloendothelial system (RES) uptake. Therefore, the conjugate of the present disclosure has improved pharmacokinetic properties such as prolonged blood circulating half-life. The conjugate of the present disclosure also has improved anticoagulant properties such as higher prothrombin time, prolonged activated partial thromboplastin time (aPTT), longer anticoagulation period, and lower risk of causing HIT. The VP28-heparin nanocomplex of the present disclosure exhibits superior anticoagulation properties to native heparin.

In an embodiment of the present disclosure, the conjugate of heparin and a viral capsid protein VP28 is formed by a covalent bond between heparin and VP28. In at least one embodiment of the present disclosure, the heparin is conjugated to the viral capsid protein by a carbodiimide reaction. In at least one embodiment of the present disclosure, the heparin is conjugated to the viral capsid protein by an amide bond between a carboxyl group of a polysaccharide chain of the heparin and an amine group of the VP28.

In an embodiment of the present disclosure, the viral capsid protein VP28 is a recombinant viral capsid protein. In at least one embodiment of the present disclosure, the VP28 forms a trimer. In some embodiments of the present disclosure, the viral capsid protein VP28 of the conjugate undergoes self-assembly. In an embodiment of the present disclosure, the self-assembly of the viral capsid protein VP28 of the conjugate is reversible. In at least one embodiment of the present disclosure, the viral capsid protein VP28 forms a virus-like particle (VLP). In at least one embodiment of the present disclosure, the conjugate of heparin and a viral capsid protein VP28 forms a nanoparticle. In at least one embodiment of the present disclosure, the conjugate of the present disclosure is designed to form a trimeric nanocomplex composed of VP28 bound to heparin.

In an embodiment of the present disclosure, the heparin of the conjugate is a heparin or a derivative thereof. In an embodiment of the present disclosure, the heparin of the conjugate is unfractionated heparin (UFH), fractionated heparin, low molecular weight heparin, or any derivative thereof. In an embodiment of the present disclosure, the heparin derivative is heparinoid, enoxaparin, dalteparin, or tinzaparin.

In an embodiment of the present disclosure, the hydrodynamic size of the conjugate is less than that of heparin. In an embodiment of the present disclosure, the hydrodynamic size of the conjugate is higher than the renal filtration threshold. In at least one embodiment of the present disclosure, the average hydrodynamic size of the conjugate is concentration-dependent. In at least one embodiment of the present disclosure, the average hydrodynamic size of the conjugate is between 3 nm and 50 nm, between 5 nm and 45 nm, between 5 nm and 40 nm, between 6 nm and 35 nm, between 8 nm and 40 nm, or between 8 and 35 nm.

In an embodiment of the present disclosure, the average surface charge of the conjugate is higher than that of heparin. In an embodiment of the present disclosure, the average surface charge of the conjugate is less negative than that of heparin.

According to an embodiment of the present disclosure, a method is provided for preparing a conjugate of heparin, wherein the heparin is conjugated to a viral capsid protein VP28.

According to an embodiment of the present disclosure, a pharmaceutical composition comprising a conjugate of heparin is provided with its therapeutic use to prevent blood coagulation. In at least one embodiment, the present disclosure provides a method of preventing or treating a condition or a disease associated with thrombosis by administering the pharmaceutical composition of the present disclosure to a subject in need thereof. In at least one embodiment of the present disclosure, the condition or the disease associated with thrombosis is myocardial infarction, pulmonary embolism, neonatal respiratory distress syndrome, adult respiratory distress syndrome, primary carcinoma of lung, non-Hodgkin's lymphoma, fibrosing alveolitis, lung transplant, atherosclerosis, surgery, stroke, and thrombosis. In at least one embodiment, the present disclosure provides a method to anti-coagulate blood in a subject when such a blood anticoagulation is needed. In at least one embodiment, the present disclosure provides a method to anti-coagulate blood in a subject during a surgery, a cardiac catheterization, a cardiopulmonary bypass, or a hemodialysis.

### BRIEF DESCRIPTION OF DRAWINGS

The present disclosure can be more understood by reading the following descriptions of the embodiments, with reference made to one or more of the accompanying drawings below.

FIGS. 1A to 1C show the results of expression, Western blotting, and His-tag column purification for VP28, respectively. FIG. 1A shows the results of SDS-PAGE, and FIG. 1B shows in the results of Western blotting analysis with VP28 obtained from different procedures. Lane M: marker proteins; lane 1: total cell lysate of non-induced cells; lane 2: total cell lysate; lane 3: soluble protein; lane 4: inclusion bodies. FIG. 1C shows the result of purification of VP28 by His-tag column. Lane 1: non-purified VP28 protein obtained from refolded VP28 from inclusion bodies; lane 2: passthrough; lane 3: column wash; lanes 4 to 8: fractions of eluted VP28 proteins.

FIGS. 2A to 2C show the circular dichroism (CD) spectra of various refolding states of VP28. FIG. 2A shows the VP28 spectra in the far-UV range from 200 to 250 nm. R1 to R5 represent each refolding stage. FIG. 2B shows the combined CD spectra from R1 to R5 at 222 nm. FIG. 2C shows the secondary structure analysis of refolded VP28 at R5 refolding stage.

FIGS. 3A to 3C show the fluorescence spectroscopy analysis of refolded VP28. FIGS. 3A and 3B show the fluorescence spectra of VP28 in different refolding buffer, i.e., denaturing buffer (U) and refolding buffers 1 to 5 (R1, R2, R3, R4, and R5), with each representing different refolding stage. FIG. 3C shows the blue shift effect of VP28 in different refolding buffers during its refolding.

FIGS. 4A to 4I show the size and charge analysis of rVP28, unfractionated heparin (UFH), and VP28-conjugated heparin (VP28-heparin). FIG. 4A shows the particle size measurement of rVP28 and rVP28-heparin complex in various concentrations (0.0625, 0.125, 0.25, and 0.5 mg/mL), where circles represent rVP28-heparin complex and squares represent rVP28. FIG. 4B shows particle size measurement of VP28 and VP28-heparin complex in 0.0625 mg/mL. FIG. 4C shows the transmission electron microscopy (TEM) image of VP28 self-assembled particles on super-thin carbon film grid. The scale bar is 50 nm. The inset is the enlarged figure of VP28 self-assembled particle. The scale bar is 20 nm. FIG. 4D shows non-staining high-resolution transmission electron microscopy (HRTEM) images of VP28 self-assembled particles on a super-thin carbon film grid. The scale bar is 10 nm. FIG. 4E shows the autocorrelation function of heparin by DLS. The squares denote the measured results, and the circles denote the non-negative least-squares (NNLS) fitting results. FIG. 4F shows the particle size distribution analysis of heparin by NNLS fitting. FIG. 4G shows zeta potential analysis for VP28, heparin, and VP28-heparin. FIG. 4H shows the TEM images of VP28-heparin particles on super-thin carbon film grid. The scale bar is 20 nm. FIG. 4I shows the HRTEM images of VP28-heparin particles on super-thin carbon film grid. The scale bar is 10 nm.

FIGS. 5A and 5B show the results of VP28 antibody detection using VP28-ELISA after intraperitoneal injection of five mice in each treatment group. In FIG. 5A, the mice received an injection of either 100 μg purified VP28 or VP28 conjugated with heparin. The anti-sera titers against VP28 protein were determined after the subcutaneous injection (12,800× dilution) on Day 0, Day 14, and Day 28. ANOVA was used to analyze the statistical significance. ***P<0.001 is considered to be statistically significant. FIG. 5B shows the HIT effect in mice by analyzing the representative total platelet count of sera from immunized mice and heparin-treated mice at different time points (Pre (pretreatment), 24 h and 7 days). Each data point represents the mean+SD. ANOVA was used to analyze the statistical significance. *P<0.05 is considered to be statistically significant when compared with pretreatment (Pre) in the same group.

FIGS. 6A and 6B show the anticoagulant effect of heparin and VP28-heparin. FIG. 6A shows the result of prothrombin time (PT), and FIG. 6B shows the result of activated thromboplastin time (aPTT). The filled squares represent heparin, and the open circles represent VP28-heparin. ANOVA was used to analyze the statistical significance. *P<0.05 and ***P<0.001 are considered to be statistically significant (n=3).

FIG. 7 shows the reversal of the anticoagulative effects of heparin and VP28-heparin by using protamine sulfate (PS).

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications. Titles or subtitles may be used in this disclosure for the convenience of a reader, which shall have no influence on the scope of the present disclosure.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “pharmaceutically acceptable vehicle” refers to a pharmaceutically acceptable material, composition, or carrier, such as diluents, disintegrating agents, binders, lubricants, glidants, and surfactants, which do not abrogate the biological activity or properties of the active ingredient (e.g., the VP28-heparin conjugate used herein), and is relatively non-toxic; that is, the vehicle may be administered to a subject without causing an undesirable biological effect or interacting in a deleterious manner with any of the components of the pharmaceutical composition in which it is contained.

As used herein, the terms “treat,” “treating,” and “treatment” refer to acquisition of a desired pharmacologic and/or physiologic effect, e.g., alleviating or abrogating a disorder, disease, or condition, or one or more of the symptoms associated with the disorder, disease, or condition, or alleviating or eradicating the cause(s) of the disorder, disease, or condition itself. The effect may be prophylactic in terms of completely or partially preventing a disease or symptom thereof or may be therapeutic in terms of completely or partially curing, alleviating, relieving, remedying, or ameliorating a disease or an adverse effect attributable to the disease or symptom thereof. Treatment also includes use of the compositions of the present disclosure associated with a medical procedure with administration before, during, or after the medical procedure.

As used herein, the terms “prevent,” “preventing,” and “prevention” refer to inclusion of a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The phrase “an effective amount” refers to the amount of an active ingredient that is required to result in a reduction, inhibition, or prevention of a disorder, disease, or condition, and/or its attendant symptoms in a subject. An effective amount will vary, as recognized by those skilled in the art, depending on routes of administration, excipient usage, and the possibility of co-usage with other therapeutic treatment.

As used herein, the terms “subject” and “individual” may be interchangeable and refer to an animal, e.g., a mammal including the human species. The term “subject” is intended to refer to both the male and female gender unless one gender is specifically indicated. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The conjugate of the present disclosure provides a heparin-nanocarrier derivative or a heparin complex with less non-specific binding to protein, cell and/or tissue, higher plasma half-life, and lower HIT risk. In an embodiment of the present disclosure, the heparin complex is a heparin-nanocarrier conjugate where the nanocarrier is a virus-like particle (VLP). As used herein, the term “virus-like particles (VLPs)” are nanomaterials ranging in size from a few nanometers to 200 nm. VLPs contain no viral genetic material and can self-assemble in vitro from original viral capsid proteins, and refer to a structure that in at least one attribute resembles a virus but which has not been demonstrated to be infectious. VLPs in accordance with the present disclosure do not carry genetic information encoding for the proteins of the VLPs. In general, VLPs lack a viral genome and, therefore, are noninfectious. In addition, VLPs can often be produced in large quantities by heterologous expression and can be easily purified. Generally, VLPs are nanomaterials having a size of 10 nm to 1,000 nm, 20 nm to 500 nm, 40 nm to 400 nm, 70 nm to 300 nm, or 80 nm to 200 nm. VLPs can be used as protein nanocarriers with biocompatibility, biodegradability, low cytotoxicity, and low immunogenicity.

Heparin is composed of negatively charged linear chains of disaccharide units of D-glucosamine and uronic acid (L-iduronic acid or D-glucuronic acid). Heparin interacts with various ligands in a charge-dependent manner. Highly-negative heparin interacts with the positively charged platelet factor 4 (PF4, CXCL4), subsequently inducing immune-mediated HIT. In addition, the interaction between heparin and PF4 is size-dependent. Therefore, a lower incidence of HIT (0.2 to 0.6%) was observed with LMWH as compared to unfractionated heparin (UFH) (HIT incidence: 2 to 3%). Unlike UFH and LMWH, fondaparinux, which is a synthetic heparin pentasaccharide, has a longer half-life and does not induce HIT.

As shown in the present disclosure, conjugating UFH to VP28 enhances UFH's physical stability and its blood circulating half-life, as well as its anticoagulation properties, whilst reducing UFH's risk in causing HIT. VP28 is a capsid protein of shrimp white spot syndrome virus (WSSV). WSSV causes white spot disease (WSD) in shrimp but is not contagious to humans. The studies of WSSV formation have indicated that the viral capsid protein formation is independent to the WSSV nucleocapsid formation. Assembly of VP28 as a VLP offers a novel nanocarrier for conjugation with heparin. In an embodiment of the present disclosure, VP28 is a recombinant protein and can be synthesized with any recombinant protein synthesis methodology known to a person skilled in the art. In an embodiment of the present disclosure, VP28 is produced by a cell transformed with a plasmid designed and constructed to express the VP28 for use in the conjugate of the present disclosure.

As provided by the present disclosure, the zeta potential of UFH was significantly reduced (2.5 times less negative than that of heparin) after its conjugation with VP28. Furthermore, the hydrodynamic size of the resultant VP28-heparin complexes was found to be between the small and large heparin fragments but slightly larger than that of VP28. The present disclosure thus provides a heparin conjugate in which both small and large heparin fragments interact closely with VP28 to form a packed nanocomplex. The VP28-heparin nanoparticle of the present disclosure therefore has a more uniformed hydrodynamic size distribution (8.81±0.58 nm) and is capable of self-assembly into larger aggregates at high nanoparticle concentrations for improved aqueous stability with a capacity for reversibly disintegrating into monomeric VP28-heparin at low nanoparticle concentrations for anticoagulative action. Such VP28-heparin complex reduces the affinity of heparin to PF4, thereby decreasing the formation of the PF4-heparin complex. This in turn reduced the induction of anti-PF4 antibodies, and the risk of immune-mediated HIT, as evidenced by the present disclosure in which the platelet count of the mice was found maintained at a normal level over 7 days after administration of VP28-heparin nanocomplexes. In comparison, HIT usually begins between 5 and 15 days after heparin administration.

### EXAMPLES

Exemplary embodiments of the present disclosure are further described in the following examples, which should not be construed to limit the scope of the present disclosure.

### Preparation Example 1. Preparation of VP28 as a Protein Nanocarrier

First, a VP28 expression plasmid was constructed by inserting the coding sequence of VP28 into the EcoRI and NcoI sites of pET30a plasmid (Thermo Fisher Scientific, USA) to generate the plasmid VP28-pET30a. VP28 was therefore cloned into the pET30a plasmid via solid-phase gene synthesis and transformed into E. coli BL21 (DE3) cells. For protein purification, the construction codes for a 6× His tag were fused with the C-terminal of VP28. Transformed E. coli BL21 (DE3) cells (Thermo Fisher, USA) were cultured in LB broth containing kanamycin (50 μg/mL) at 37° C. until the cell density reaches an O.D. of 0.3 to 0.6. Thereafter, β-D-thiogalactopyranoside (IPTG) was added to the medium at a final concentration of 1 mM, and the culture medium was incubated for 24 h. Bacterial cells were harvested by centrifugation (HERMLE, Model: Z 323K, Germany) at 10,000 rpm for 15 minutes at 5° C. The cell pellet was then resuspended in 50 mM Tris-HCl (pH 7.5), and the cells were broken using the cell disruptor (Constant Systems, Model: BT40/TS2/BA, England).

For VP28 solubilization, inclusion bodies from the cell pellet were washed three times with 50 mM Tris-HCl (pH 7.5) and then solubilized in a denaturing buffer containing 4.5 M urea (13.5 g), 10 mM Tris-base (60 mg), 0.1 M 2-mercaptoethanol (383 μL), 0.1% mannitol (50 mg), and 0.1 μM Pefabloc (5 μL) overnight. The solubilized VP28 was centrifuged at 10,000 rpm for 30 minutes to remove insoluble proteins.

For VP28 refolding, five different refolding buffers were used to gradually remove urea for correct refolding. The denaturing and refolding buffers and conditions used were shown in Table 1 below.

TABLE 1
Buffers and conditions used for denaturing and refolding

| | | Tris- | | | | | | | Buffer |
|---|---|---|---|---|---|---|---|---|---|
| | Urea | base | 2-Me | Mannitol | Pefabloc | | Temp. | Volume | change |
| Buffer | (M) | (mM) | (M) | (%) | (μM) | pH | (° C.) | (L) | times/h |
| Denaturing | 4.5 | 10 | 0.1 | 0.1 | 0.1 | 11 | 4 | 0.004 | 1/12 |
| Refolding 1 | 2 | 10 | 0.1 | 0.1 | 0.1 | 11 | 4 | 2.5 | 2/48 |
| Refolding 2 | 1 | 10 | 0.1 | 0.1 | 0.1 | 11 | 4 | 2.5 | 3/24 |
| Refolding 3 | 0 | 10 | 0.1 | 0.1 | 0.1 | 11 | 4 | 2.5 | 3/24 |
| Refolding 4 | 0 | 10 | 0.1 | 0.1 | 0.1 | 8.8 | 4 | 2.5 | 3/24 |
| Refolding 5 | 0 | 10 | 0.1 | 0 | 0.1 | 8.8 | 4 | 2.5 | 3/24 |

2-Me: 2-mercaptoethanol

Solubilized VP28 was placed within a dialysis buffer containing the first refolding buffer (2.5 L, pH 11 to 12) consisting of 2 M urea (300 g), 10 mM Tris-base (3 g), 0.1 M 2-mercaptoethanol (19 μL), 0.1% mannitol (2.5 g), and 0.1 μM Pefabloc (250 μL) and dialyzed for two days. Thereafter, VP28 was dialyzed against the second refolding buffer (2.5 L, pH 11 to 12), which consists of 1 M urea (150.15 g), 10 mM Tris-base (3 g), 0.1 M 2-mercaptoethanol (19 μL), 0.1% mannitol (2.5 g), and 0.1 μM Pefabloc (250 μL) for one day. Subsequently, VP28 was dialyzed in the third refolding buffer (containing no urea) for one day. The composition of the third refolding buffer (2.5 L, pH 11 to 12) was 10 mM Tris-base (3 g), 0.1 M 2-mercaptoethanol (19 μL), 0.1% mannitol (2.5 g), and 0.1 μM Pefabloc (250 μL). Lastly, VP28 was dialyzed in the fourth refolding buffer (2.5 L, pH 8.8), which contains 10 mM Tris-base (3 g), 0.1 M 2-mercaptoethanol (19 μL), 0.1% mannitol (2.5 g), and 0.1 μM Pefabloc (250 μL) for one day.

The purification of the His-tagged VP28 protein was performed by affinity chromatography. A column with 1 mL of His-Trap resin (GE Healthcare) was equilibrated with 10 volume columns of a binding buffer (25 mM Tris-HCl, 40 mM imidazole, 500 mM NaCl, pH 7.4) and loaded with a sample containing the His-tagged VP28 protein. The column was washed with 10 volume columns of a binding buffer (25 mM Tris-HCl, 40 mM imidazole, 500 mM NaCl, pH 7.4). His-tagged VP28 was eluted with an elution buffer (25 mM Tris-HCl, 500 mM imidazole, 500 mM NaCl, pH 7.4) and collected in 1.5 mL fractions. Sodium dodecyl sulfate polyacrylamide gel electrophoresis (SDS-PAGE) was performed to detect the fraction containing the VP28 protein. Fractions containing the VP28 were then dialyzed in 50 mM Tris-HCl, pH 7.5 to remove imidazole.

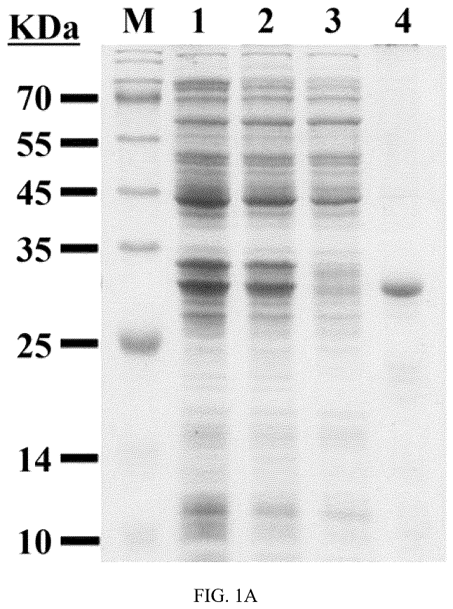

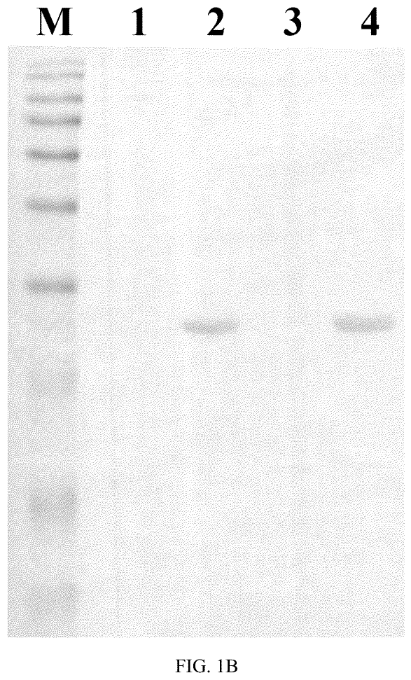

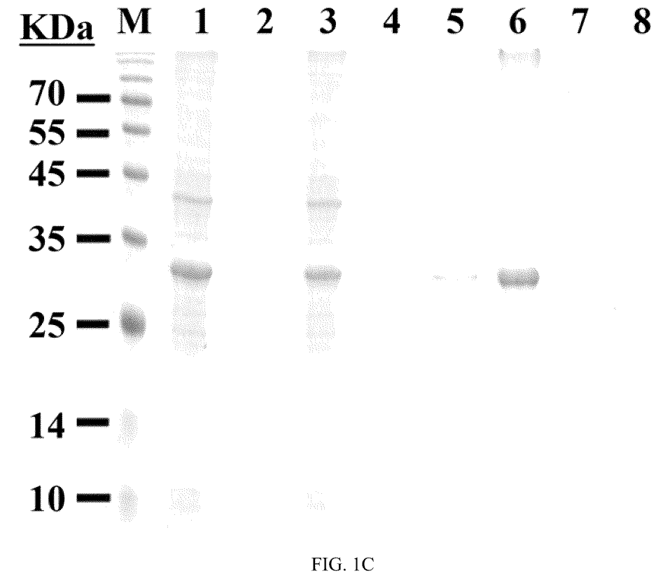

To assess the presence and purity of the recombinant VP28 obtained from different procedures, it was resolved in 15% SDS-PAGE and analyzed by Western blotting, and the result was shown in FIGS. 1A to 1C. The obtained recombinant VP28 was first resolved in 15% SDS-PAGE, followed by Coomassie blue staining and subsequently electrophoretic transfer to polyvinylidene difluoride (PVDF) membrane (wet transfer, 130 V, 180 minutes). Tris-buffered saline (TBS) containing 5% non-fat dry milk was used to block the PVDF membrane for 1 h at room temperature. Excess blocking buffer was removed by washing the PVDF membrane with TBS for 10 minutes. The PVDF membrane was then incubated with the primary antibody (His-Tag Rabbit polyclonal Ab, Affinity Biosciences, USA) in a dilution ratio of 1:25,000 at 4° C. overnight. Thereafter, the PVDF membrane was washed 3 times by TBST (Tris-buffered saline with Tween 20) for 10 minutes at room temperature, followed by further incubation with the secondary antibody (alkaline phosphatase-conjugated affinity pure goat anti-rabbit IgG (H+L); Jackson ImmunoResearch, USA) (1:25,000) for 1 hr at room temperature. The PVDF membrane was then washed 3 times by TBST for 10 minutes per wash at room temperature. Detection was achieved using kits containing 5-bromo-4-chloro-3-indolyl phosphate (BCIP)/nitro blue tetrazolium (NBT) (Thermo Scientific, USA) with an alkaline phosphatase (ALP) secondary antibody.

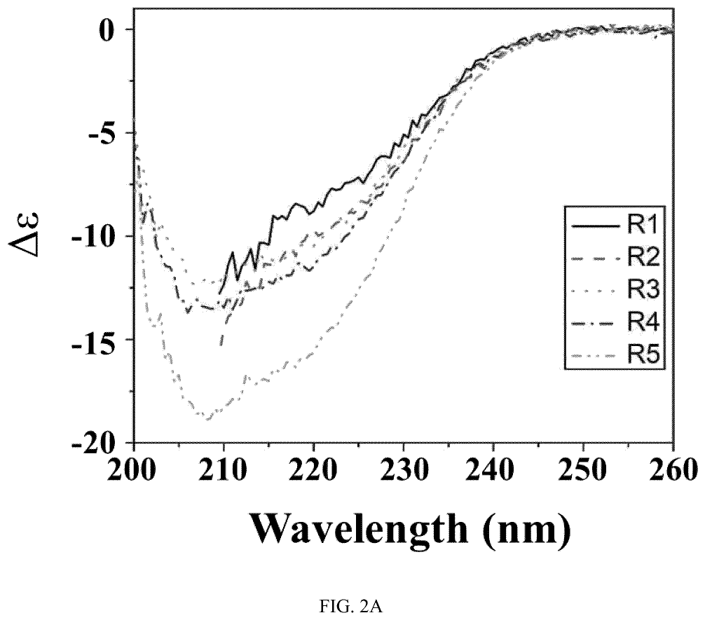

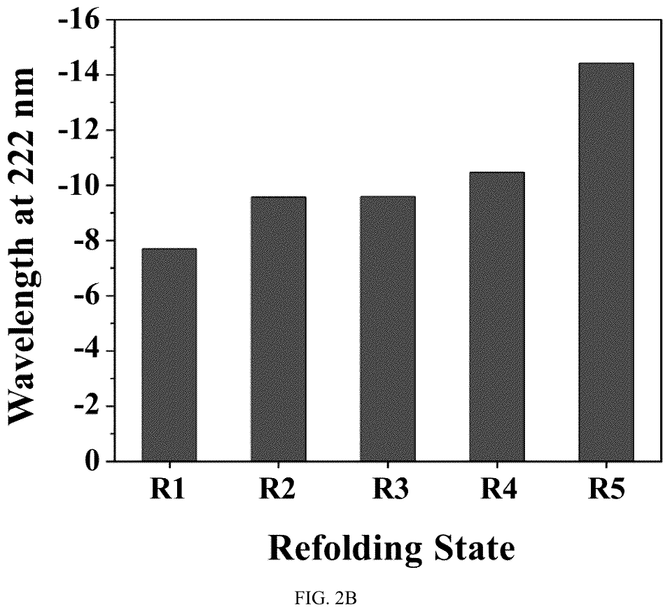

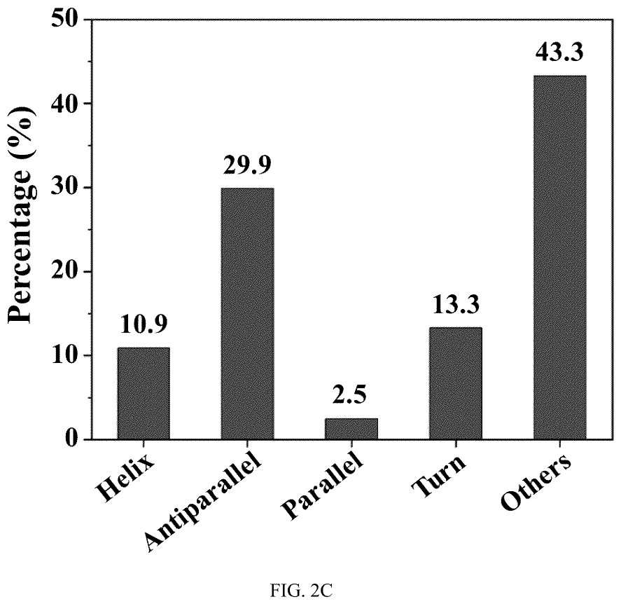

The synthesized recombinant VP28 protein nanocarrier (rVP28) was determined to have a molecular weight of 30 kDa, as shown in FIGS. 1A to 1C, which was in agreement with previous studies. Then, circular dichroism (CD) measurements for analysis on the secondary structure of the recombinant VP28 were conducted using Jasco (J-815-150S) spectropolarimeter (Jasco Ltd., Tokyo, Japan; Model: J-815, Serial No. B055861168). For instance, purified proteins (VP28) were dissolved in 20 mM Tris-HCl buffer at pH 8 to obtain a protein concentration of 0.1 mg/mL. Spectra were recorded in the far ultraviolet (UV) region (200 to 260 nm). Protein samples were measured at room temperature in a 1 mm path length quartz cell (350 μL) using a scan speed of 50 nm/min, a time response of 1 second, and a bandwidth of 1 nm. Analysis was carried out on an average of three scans. As shown in FIGS. 2A to 2C, the VP28 obtained from above consists of around 10.9% α-helices, 29.9% anti-parallel β-sheet, 2.5% parallel β-sheet, 13.3% turns, and 43.3% other structures. This was corroborated by the crystal structure of the VP28 trimer previously reported, such as in a report by Wu et al. (J. Virol. 81 6709-17).

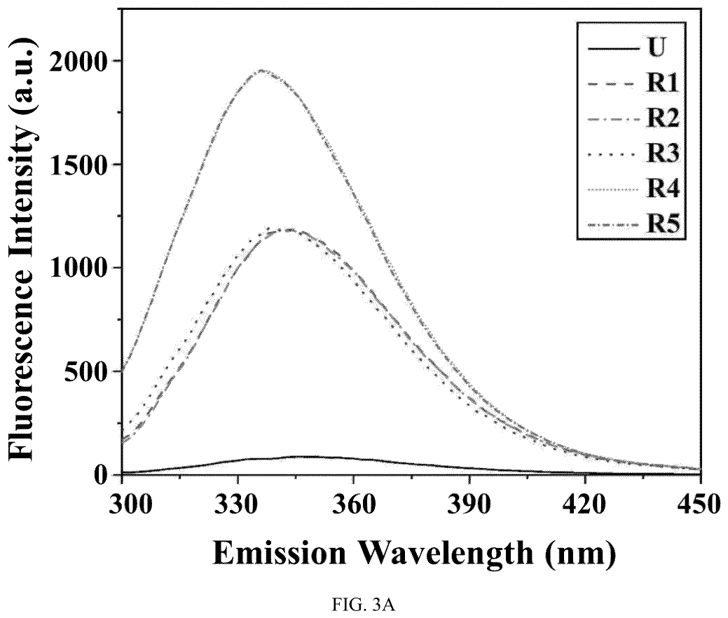

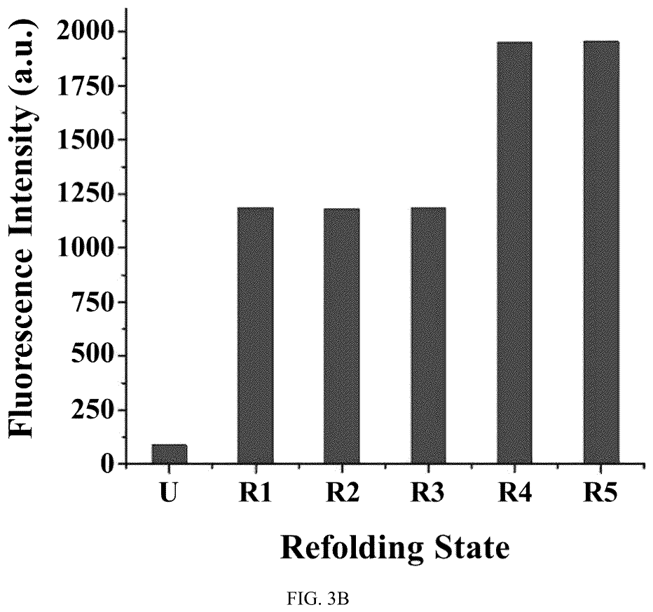

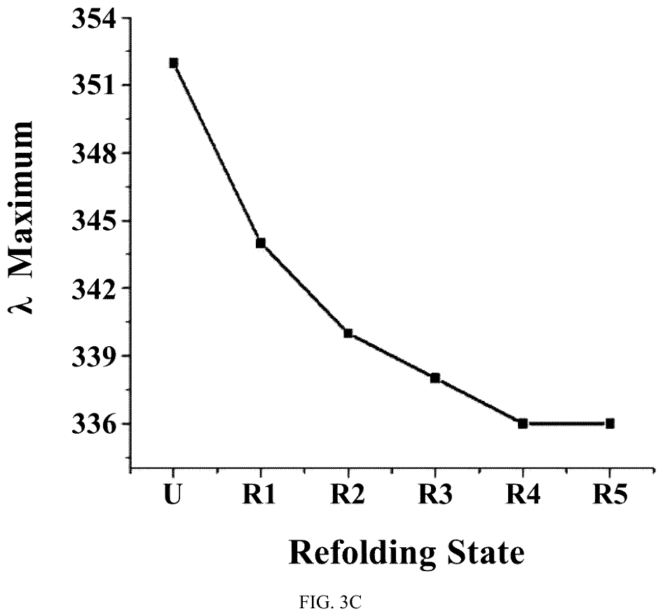

To assess the refolding state of the rVP28, intrinsic fluorescence analysis was carried out. Following each step of refolding, the intrinsic fluorescence of VP28, which was emitted by aromatic residues, tryptophan (Trp), phenylalanine (Phe), and tyrosine (Tyr), was measured at 25° C. using 1.0 cm cuvette and a Hitachi F-7000 fluorescence spectrophotometer (Hitachi High Technologies, Tokyo, Japan). To determine intrinsic fluorescence of VP28, an excitation wavelength of 280 nm was used, and the emitted spectra were recorded in the range of 300 nm to 450 nm. The fluorescence spectroscopy analysis results are shown in FIGS. 3A to 3C, which revealed an escalation in the fluorescence emission (FIGS. 3A and 3B) and a blue shift of maximum emission wavelength from 352 to 344, 340, 338, 336, and 336 nm (FIG. 3C). These changes in the VP28's fluorescence emission spectra suggested the successful folding of VP28 into a stable tertiary conformation that consists of a hydrophobic core.

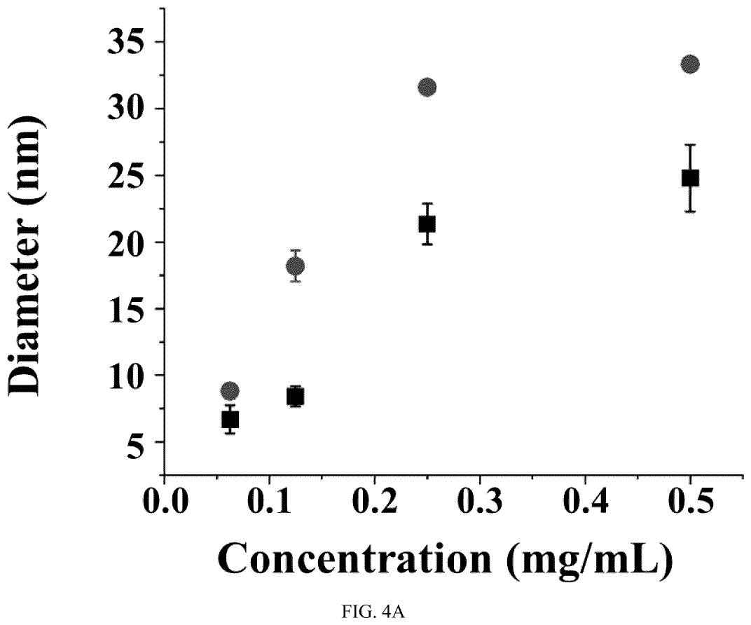

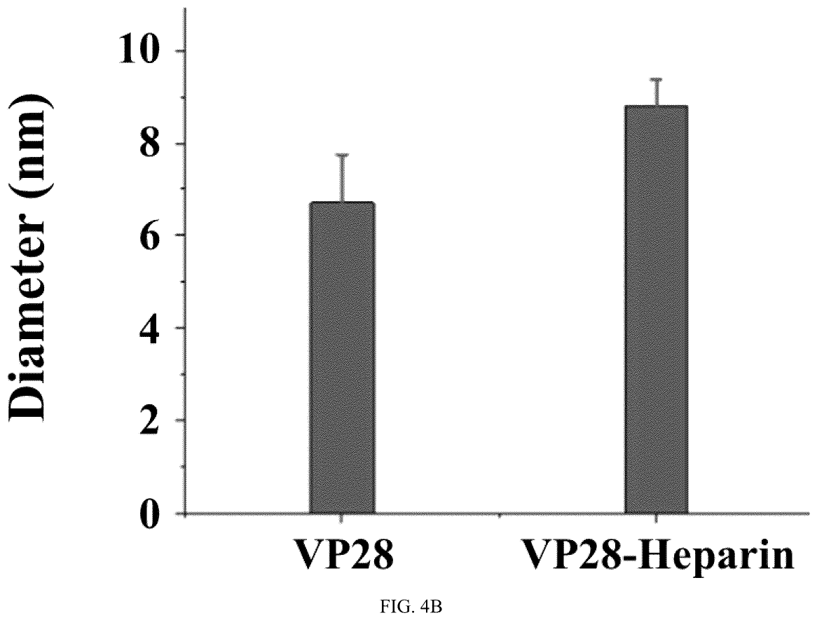

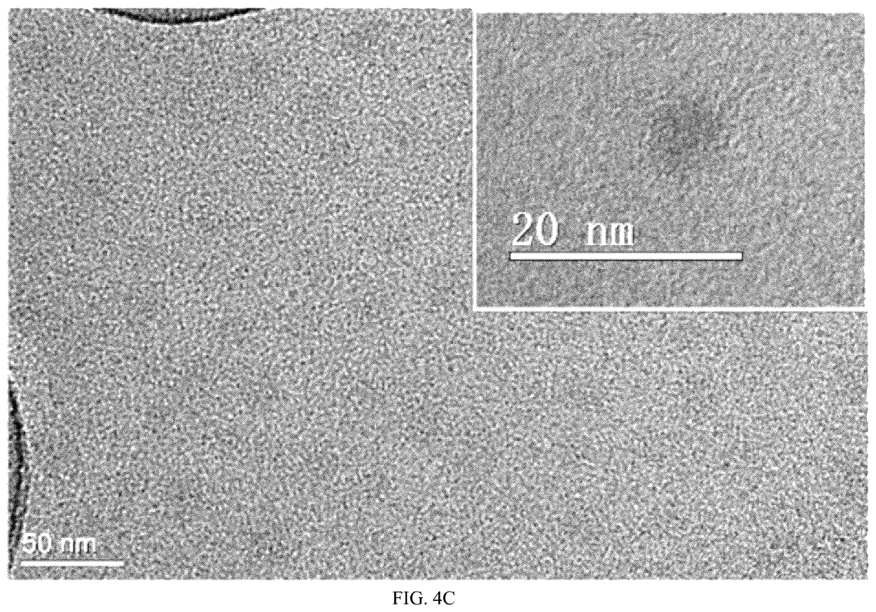

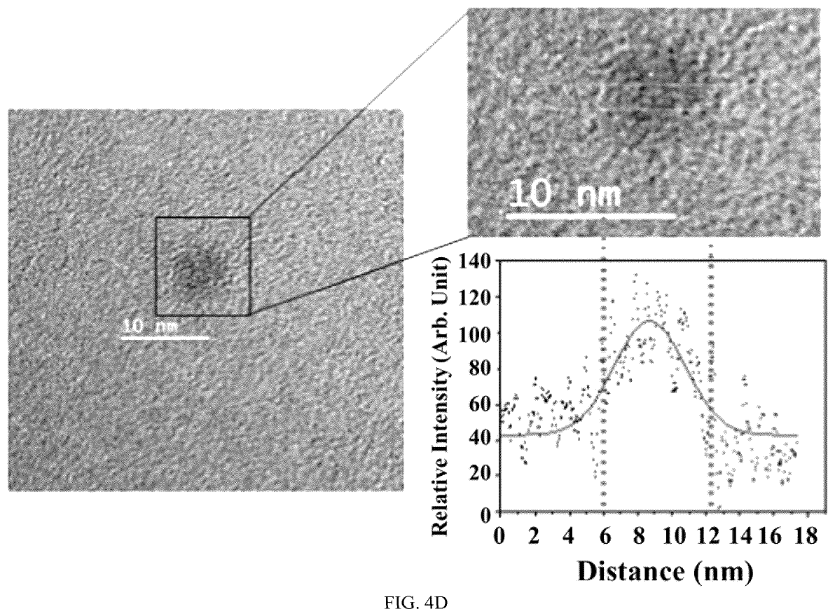

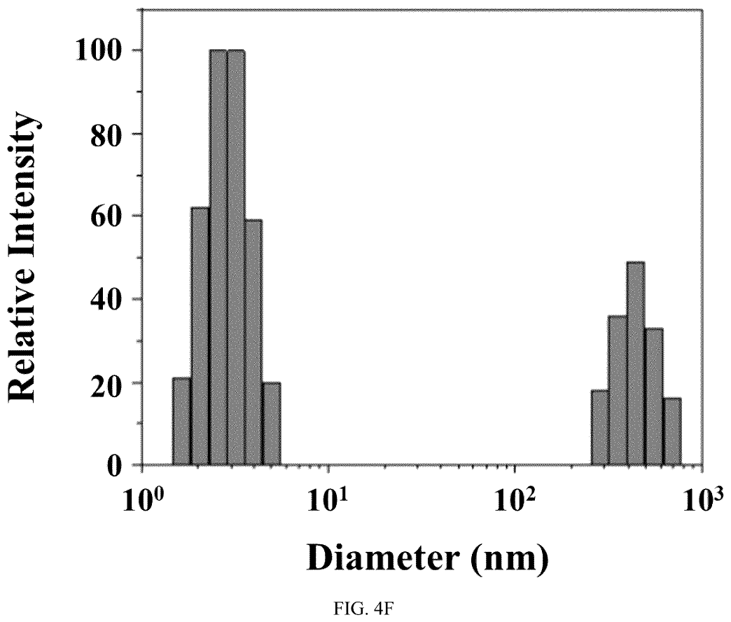

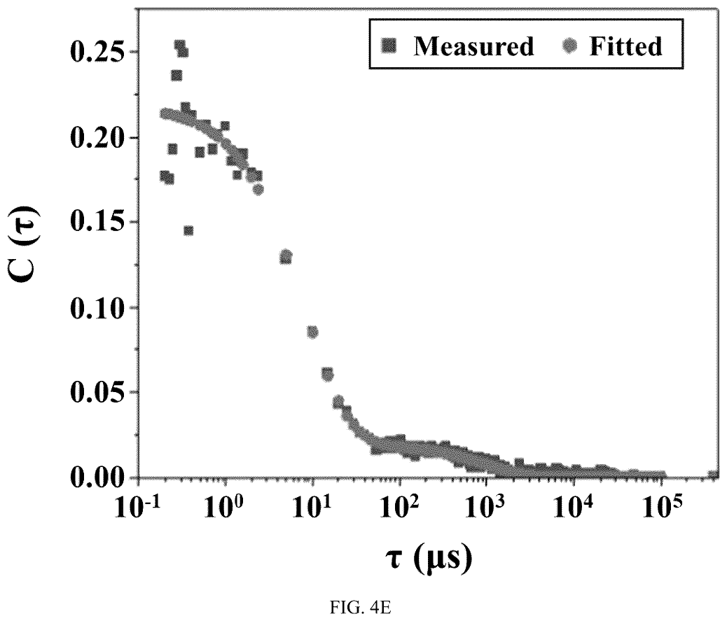

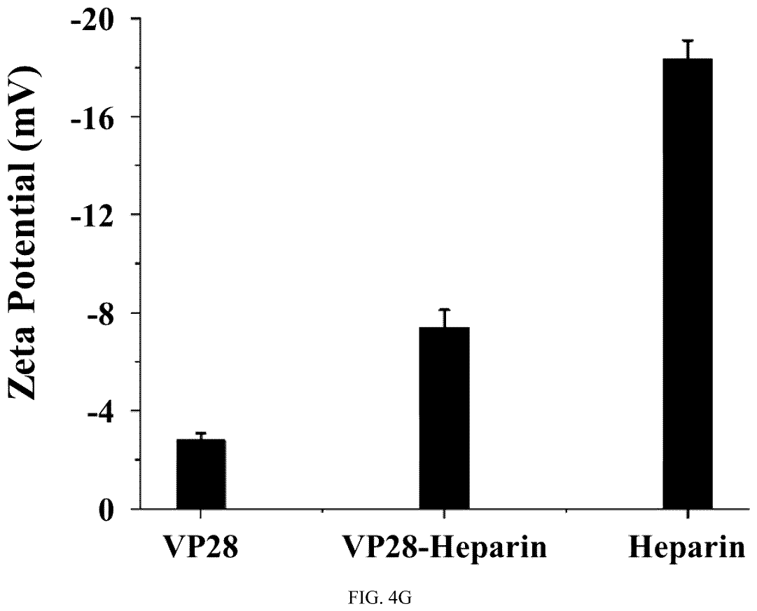

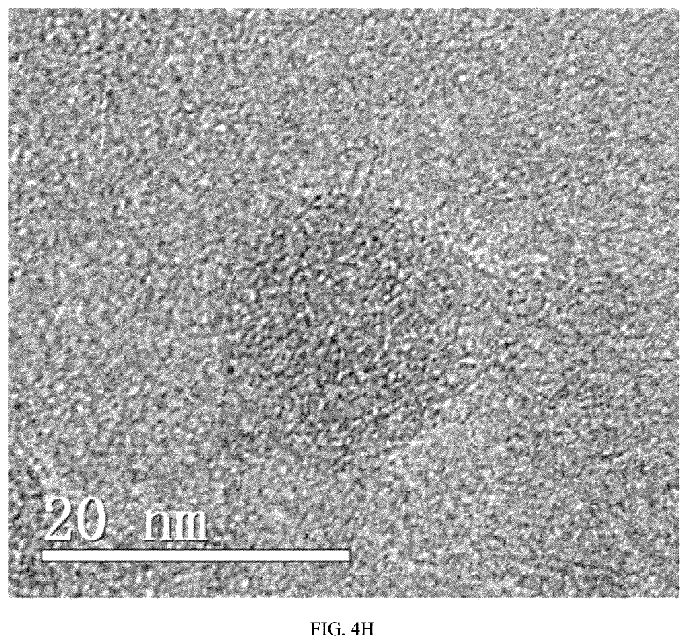

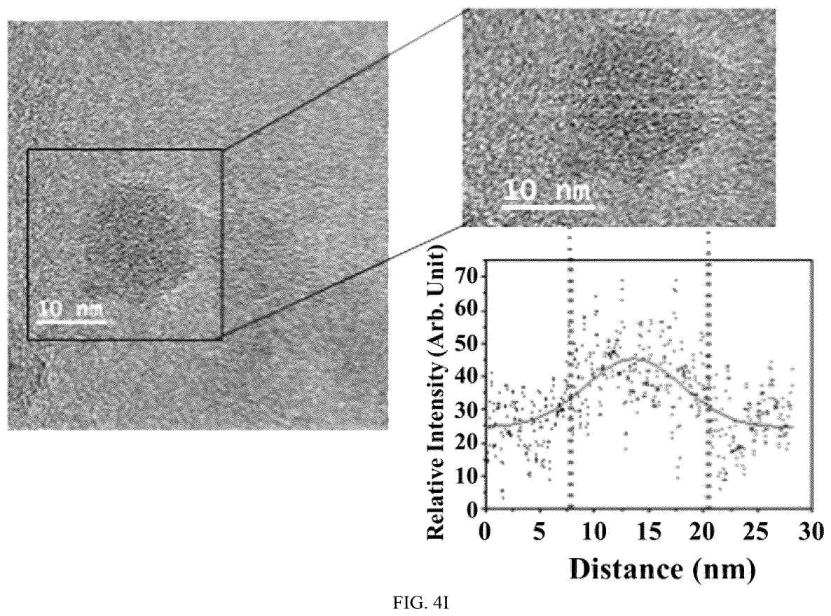

For measuring self-assembled particles of rVP28, the size distribution was measured by dynamic light scattering (DLS) using goniometer (Brookhaven, Inc., Holtsville, NY), including a diode-pumped laser with power of 100 mW and a wavelength of 532 nm (Coherent, Santa Clara, CA). At 90°, the scattered light was collected from the incident direction. The chamber temperature was around 20° C., and the sample volume was 3 mL. Digital correlator (BI 9000, BIC, Holtsville, NY) was used to calculate the autocorrelation function, which was then analyzed by the negatively constructed least-squares method. As shown in FIG. 4A, rVP28 at 0.5 mg/mL had an average hydrodynamic size of 24.8±2.5 nm and a zeta potential of −2.8±0.28 mV. The average hydrodynamic size was found to gradually reduce to 6.7±1.06 nm after rVP28 suspension was diluted to a concentration of 0.0625 mg/mL (FIGS. 4A and 4B). FIG. 4B shows the particle size of rVP28 and VP28-heparin complex measured in 0.0625 mg/mL.

Alterations of the rVP28 average hydrodynamic size at different rVP28 concentrations suggested the presence of concentration-dependent and reversible self-assembly of rVP28 in aqueous environments. rVP28 self-assembled into larger nano-complexes at the higher rVP28 concentration (0.5 mg/mL, FIG. 4A), whereas the nano-complexes gradually disintegrated into smaller aggregates or rVP28 trimeric units following a decrease in the rVP28 concentration. The concentration-dependent and reversible self-assembly of rVP28 was further confirmed by the visualization of rVP28 nano-complexes of various sizes (around 15 to 24.8 nm, prepared from 0.5 mg/mLrVP28 suspension) under non-staining atomic resolution transmission electron microscopy (TEM), as shown in FIGS. 4C and 4D.

### Preparation Example 2. Preparation of VP28-Heparin Conjugate

VP28 was covalently bonded to heparin through amide bonds by reacting the amine group of VP28 and the carboxyl group of heparin using ethylene dichloride (EDC). 300 μL of VP28 (1 mg/mL) were mixed with 200 μL (1,000 units) of heparin sodium (commercially available as Agglutex injection from Chunghwa Senior Care Co., Ltd.) and 50 μL of EDC. The final aliquot was incubated at 4° C. overnight on a rotary shaker. The differences in the surface charge of VP28, heparin, and the fabricated VP28-heparin were determined (ten runs) using a 90Plus zeta potential analyzer (Brookhaven Instrument, Model: ZetaPALS, USA).

Different concentrations of VP28 (0.5, 0.25, 0.125, and 0.0625 mg/mL) were reacted with heparin at 50 mM Tris-HCl, pH 7 at 4° C. overnight via the method described above. At the same time, different concentrations of VP28 (0.5, 0.25, 0.125, and 0.0625 mg/mL) were incubated in 50 mM Tris-HCl, pH 7 at 4° C. overnight. The incubation products from both experiments were subjected to size analysis by DLS as described above. The result is shown below in the characterization of heparin and VP28-conjugated heparin (VP28-heparin).

### Example 1. Characterization of Heparin and VP28-Conjugated Heparin (VP28-Heparin)

First, unfractionated heparin (UFH) was found to exist in 2 molecular sub-fractions respectively, with average hydrodynamic sizes of 3.0±0.8 nm and 452.7±118.4 nm, as shown in FIGS. 4E and 4F, and an average charge of −18.35±0.75 mV, as shown in FIG. 4G.

UFH was chemically conjugated to the rVP28 trimer to yield a VP28-heparin nanoparticle, through the formation of amide bonds between the carboxyl groups of the heparin polysaccharide chain and the amine group of VP28, via the carbodiimide reaction. The VP28-heparin nanocomplexes formed were also shown to exhibit concentration-dependent and reversible self-assembly properties resembling that of the rVP28, as depicted by the occurrence of concentration-dependent decrease in the average hydrodynamic size of VP28-heparin nanocomplexes in an aqueous environment, from 33.3±0.23 nm at 0.5 mg/mL to 8.81±0.58 nm at 0.0625 mg/mL, as shown in FIGS. 4A and 4B. The surface charge of the VP28-heparin complexes was relatively higher (less negative) than that of the heparin but relatively lower (more negative) than that of the rVP28, as shown in FIG. 4G, indicating the charge alteration following the successful complexing of the heparin to rVP28. One VP28-heparin complex population with an average hydrodynamic size of 8.81±0.58 nm and average surface charge of −7.42±0.7, as shown in FIGS. 4A, 4B and 4G, was produced following the chemical conjugation of heparin (regardless of the molecular sub-populations) to rVP28. The average particle size of 8.81±0.58 nm was also confirmed under non-staining atomic resolution transmission electron microscopy (TEM), as shown in FIGS. 4H and 4I.

The average hydrodynamic size of the VP28-heparin complexes (8.81±0.58 nm) was found slightly elevated when compared to that of the small-sized heparin sub-fraction (average hydrodynamic size: 3.0±0.8 nm) and rVP28 (average hydrodynamic size: 6.7±1.06 nm), but significantly smaller than that of the large-sized heparin sub-fraction (average hydrodynamic size: 452.7±118.4 nm). This suggests the occurrence of close interaction between the rVP28 tertiary structure and both the short-chain heparin and long-chain heparin sub-fractions, giving rise to a VP28-heparin complex with packed conformation, as reflected by the unexpectedly small elevation in the average hydrodynamic size of the VP28-heparin complexes produced from the chemical conjugating reactions of the rVP28 and UFH.

The proposed packed conformation and lower negative surface charge of the VP28-heparin complexes reduce their non-specific interactions with the plasma proteins and platelet/endothelial-secreted proteins (e.g., PF4 and vWF). This helps to minimize the fluctuations in the plasma levels and the anticoagulative effects of heparin in the patients during treatment, as well as to reduce the risk of HIT (due to reduced binding to PF4).

Previously, it has been shown that unfractionated heparins administered were mainly cleared by the body's reticuloendothelial system (RES) and vascular endothelial cells, while the low molecular weight heparins (LMWH) were mainly removed by renal excretion. In this embodiment of the present disclosure, the VP28-heparin conjugate with an average hydrodynamic size (8.81±0.58 nm) that is above the renal filtration threshold (6 to 8 nm) but smaller than that of the large-sized fraction of the UFH (average hydrodynamic size: 452.7±118.4 nm) and reduced tissue interaction (due to elevated surface charge) will have lower renal and RES clearance, therefore resulting in an extended plasma half-life and higher bioavailability of conjugated heparin.

### Example 2. Immunogenicity Analysis of VP28 and VP28-Heparin

In assessing the immunogenicity of VP28 and VP28-heparin, the development of antibodies against VP28 and VP28-heparin in the BALB/c mice receiving 100 μg (intraperitoneal injection) of pure VP28 and VP28-heparin, respectively, was assessed via analyzing the plasma samples collected from the mice at different time intervals post-administration of VP28 and VP28-heparin using enzyme-linked immunosorbent assay (ELISA; with VP28 as the immobilized probe).

In this example, six-week-old male BALB/c mice were obtained from National Laboratory Animal Center (Taipei, Taiwan) and maintained on a 12 hr light/dark cycle with controlled temperature (22±2° C.) and humidity (55±10%) in a specific pathogen-free animal facility and given ad libitum access to rodent diet (5010, LabDiet, USA) and water throughout the study. All procedures were approved by the Institutional Animal Care and Use Committee of Academia Sinica (approved protocols no. 21-09-1714) and adhered to the guidance for the Use of Laboratory Animals (National Academy Press, Washington, DC).

Then, five mice in each group were subcutaneously injected with 100 μg purified recombinant VP28 (rVP28), UFH, or VP28-heparin as antigen, respectively. Another group of 5 mice was subcutaneously immunized with 100 μL of an emulsion containing 100 μg of rVP28 in complete Freund's adjuvant. After 14 days, each group of the mice was boosted with 100 μg of rVP28, UFH, and VP28-heparin. Fourteen days after the final immunization (Day 28), ELISA was used to determine the titer of VP28 antibody. Briefly, 100 μL of VP28 (1 μg/mL) were immobilized in 96-well plates at 4° C. overnight. The wells were then washed three times with TBST buffer, followed by blocking with 200 μL of 2% bovine serum albumin (BSA) for 2 hours at room temperature and another three times of washing with TBST buffer. Next, 100 μL of diluted serum samples (the serum sample was taken from mice at Day 0, Day 14, and Day 28 post receiving VP28, UFH, VP28-heparin, and VP28 with an adjuvant) were added and incubated overnight at 4° C. The wells were then washed three times with TBST buffer. Subsequently, 100 μL of secondary labeled antibody (goat anti-mouse IgG, Bio-Rad, USA) in ratio of 1:5,000 were added to the wells, and the wells were further incubated for 2 hours at room temperature, followed by washing for three times with TBST. Lastly, 100 μL of substrate solution (Bio-Rad, USA) were added to the wells, and the wells were incubated for 30 minutes. At the end of the incubation, the absorbance of the well content was measured at 450 nm with an ELISA reader.

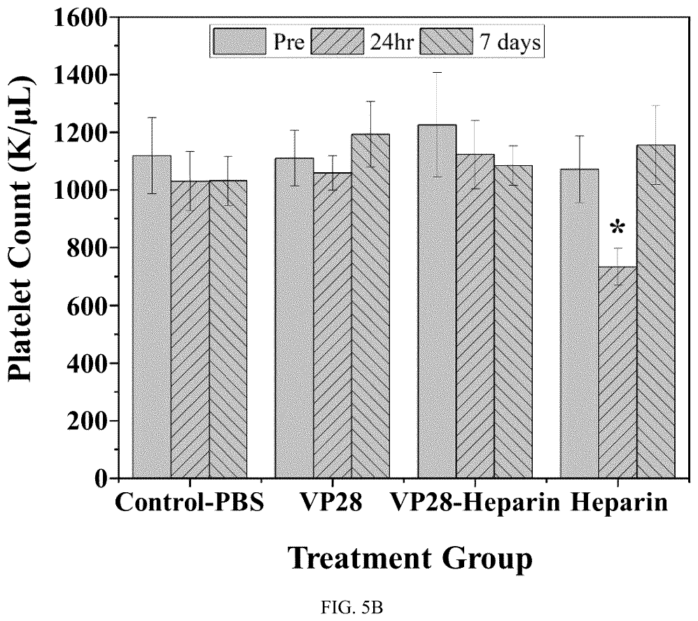

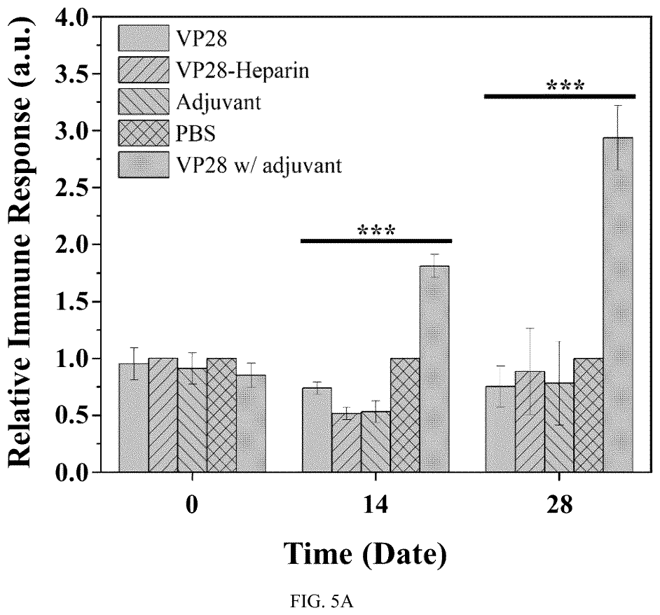

Results showed that both VP28 and VP28-heparin did not elicit the production of anti-VP28 antibodies during the 28-day experimental period (shown in FIG. 5A), in contrast to the induction of anti-VP28 immune response in mice receiving VP28 mixed with an adjuvant. This suggests the immune-inertness of both VP28 and VP28-heparin, establishing the capability of VP28 in immune-stealthing the heparin.

For platelet count of hematological analysis, tail vein blood in the presence of ethylenediaminetetraacetic acid (EDTA) was collected for hematological parameters using a Cell-Dyn 3700 hematology counter (Abbott, USA). It was found that the intravenous administration of VP28-heparin did not reduce the platelet count of the mice over a 7-day monitoring period, as opposed to the observation of platelet count reduction at Day 1 post heparin administration, as shown in FIG. 5B. This observation supports that the VP28-bond heparin is less likely to induce HIT, possibly due to reduced interactions of heparin with PF4 post complexation with VP28.

### Example 3. Anticoagulant Effect of VP28-Heparin

To assess the anticoagulant effect of VP28-heparin, both prothrombin time (PT) and active thromboplastin time (aPTT) were evaluated, as both are important coagulation testes for screening patients for bleeding tendency. The PT measures the extrinsic pathway of coagulation, and the aPTT measures the intrinsic pathway of coagulation.

Briefly, twelve-week-old Sprague-Dawley (SD) rats were used to test the anticoagulant effect of VP28-heparin. The rats were separated into experimental and control groups (n=3/group). Among the experimental groups, the animals were divided into 2 groups and injected with commercial heparin, an UFH (commercially available as Agglutex injection from China Chemical and Pharmaceutical Co., Ltd.), and VP28-heparin, respectively. To collect blood samples from the right atrium, a catheter was implanted in the right external jugular vein of adult rats. Blood (around 600 μL) was collected in 2.8% sodium citrate (9:1, v/v) for aPTT measuring before and 2, 3, 4, and 5 hours after intravenous administration in a bolus of 350 U/kg of heparin and equivalent heparin of VP28-heparin. All samples were centrifuged for 15 min at 3,500 rpm at room temperature. In sodium citrate plasma, the international normalized ratio (INR), the prothrombin time (PT), and the activated partial thromboplastin time (aPTT) were measured according to routine protocols using the ACL-TOP 750 LAS coagulation analyzer (Werfen, MA, USA).

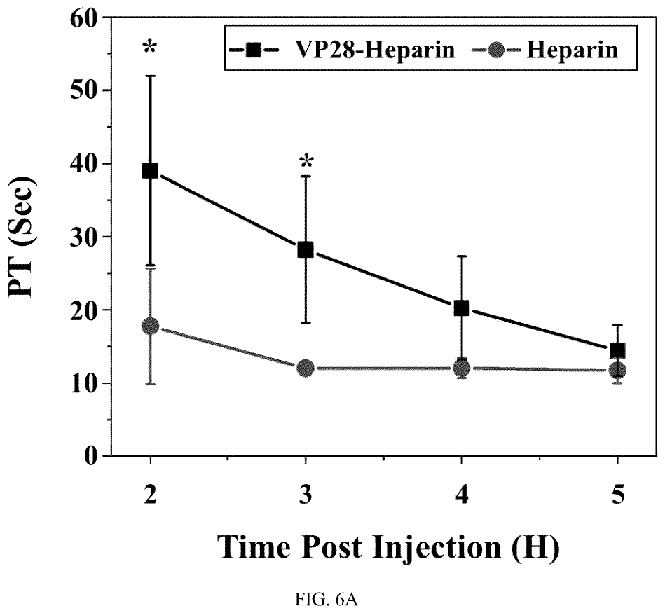

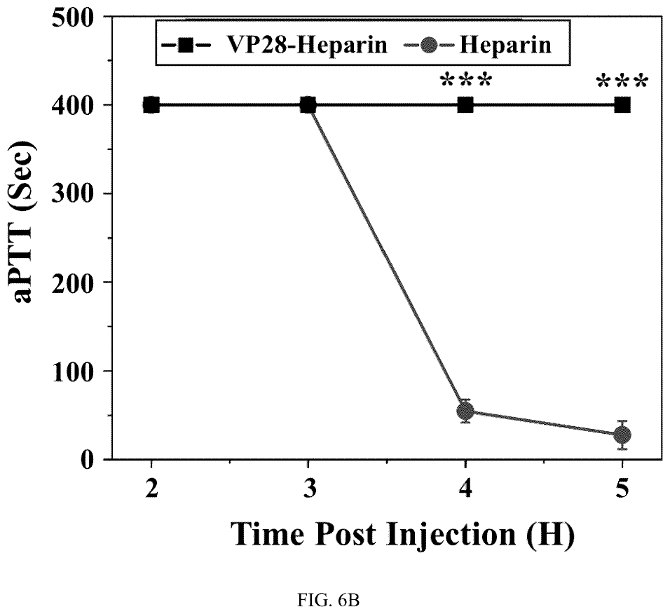

As a result, the administration of VP28-conjugated heparin to the SD rats resulted in around 2.2 times higher PT (PT_{VP28-Heparin}=39.03±12.95 s) than that caused by the administration of heparin (PT_{Heparin}=17.8±8.25 s, FIG. 6A) at 2 hours post treatment. The duration of action of VP28-heparin (5 hr) on the extrinsic pathway of coagulation was also found to be longer than that of the native heparin (2 hr, FIG. 6A). Overall, VP28-conjugated heparin was found to possess superior properties than the native heparin in attenuating extrinsic pathway coagulation.

Meanwhile, administration of heparin resulted in an elevation of aPTT to 400 s, as shown in FIG. 6B. Such observations were found to coincide well with heparin's effects on aPTT documented previously. The administration of VP28-heparin was found to elevate the aPTT to a level similar to that of the heparin administration. This indicated the conservation of the heparin's pharmacological effects on the intrinsic pathway coagulation. Surprisingly, the VP28-heparin exhibited even longer duration of action of up to 5 hours with aPTT maintained at 400 s, compared to the commercial heparin which showed a rapid decrease of aPTT and was lowered to less than 100 s at 4 hours after injection (FIG. 6B). This indicates the superior anticoagulative performance of VP28-heparin conjugate over the native heparin.

In addition, the anticoagulative effects of the VP28-heparin can be reversed using protamine sulfate (PS). To evaluate the reversal of the anticoagulative effects of VP28-heparin, protamine sulfate (MedChemExpress, NJ, USA) was used as an antidote to determine its neutralization efficacy on heparin, following the method reported previously by Ranasinghe T. et al. (J. Stroke Cerebrovascular Dis. 28), with 1 mg of protamine sulfate able to neutralize 150 to 200 U of heparin. For instance, six-week-old male BALB/c mice were divided into four experimental and control groups, with 3 mg of protamine sulfate to neutralize 400 U/kg of both heparin and heparin conjugated with VP28 in groups of BALB/c mice (n=4/group), which were administered via tail vein with either heparin or VP28-heparin. Approximately 100 μL of blood were collected in a 2.8% sodium citrate solution (in a 9:1 ratio, v/v) for aPTT measurements. This collection took place 2 h after tail vein administration of 400 U/kg of heparin and an equivalent amount of heparin from VP28-heparin. Blood samples were centrifuged for 15 min at 3,500 rpm at room temperature to obtain plasma for aPTT measurements.

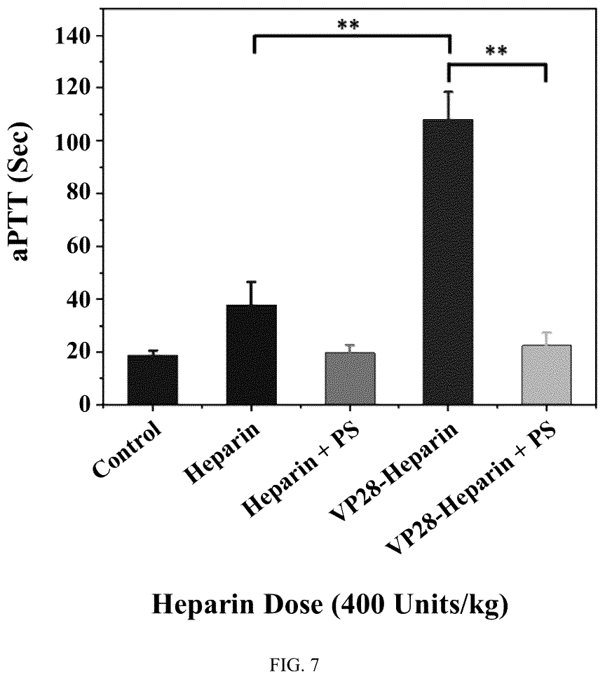

It was found that the anticoagulative effects of the VP28-heparin can be reversed using protamine sulfate in a more efficient manner compared to that of the UFH (P<0.01, FIG. 7), whereby the aPTT of VP28-heparin was reduced by approximately 4.8 folds (108.13±10 to 22.5±4.9 s) in the presence of protamine sulphate. Such reduction is greater when compared to the reversibility of the heparin's anticoagulation effects by protamine sulphate (1.9 folds, aPTT reduced from 37.7±8.7 to 19.7±3 s).

While some of the embodiments of the present disclosure have been described in detail in the above, it is, however, possible for those of ordinary skill in the art to make various modifications and changes to the embodiments shown without substantially departing from the teaching of the present disclosure. Such modifications and changes are encompassed in the scope of the present disclosure as set forth in the appended claims.

What is claimed is:

1. A conjugate of heparin and a viral capsid protein, wherein the viral capsid protein is VP28.

2. The conjugate of claim 1, wherein the heparin is conjugated to the viral capsid protein by a covalent bond.

3. The conjugate of claim 2, wherein the heparin is conjugated to the viral capsid protein by a carbodiimide reaction.

4. The conjugate of claim 2, wherein the heparin is conjugated to the viral capsid protein by an amide bond between a carboxyl group of a polysaccharide chain of the heparin and an amine group of the VP28.

5. The conjugate of claim 1, wherein the viral capsid protein is a self-assembled protein.

6. The conjugate of claim 5, wherein the self-assembly is reversible.

7. The conjugate of claim 1, wherein the viral capsid protein is a trimer.

8. The conjugate of claim 1, wherein the viral capsid protein forms a virus-like particle.

9. The conjugate of claim 1, wherein the conjugate has a hydrodynamic size less than a hydrodynamic size of heparin.

10. The conjugate of claim 9, wherein the conjugate has an average hydrodynamic size between 3 nm and 50 nm.

11. The conjugate of claim 9, wherein the conjugate has an average hydrodynamic size higher than a renal filtration threshold.

12. The conjugate of claim 11, wherein the average hydrodynamic size of the conjugate is concentration-dependent.

13. The conjugate of claim 1, wherein the conjugate has an average surface charge less negative than an average surface charge of heparin.

14. The conjugate of claim 1, wherein the heparin is unfractionated heparin (UFH), fractionated heparin, low molecular weight heparin, or a derivative thereof.

15. The conjugate of claim 14, wherein the derivative of heparin is heparinoid, enoxaparin, dalteparin, or tinzaparin.

16. A pharmaceutical composition comprising the conjugate of claim 1 and a pharmaceutically acceptable vehicle thereof.

17. A method for preventing thrombogenesis, comprising administering the pharmaceutical composition of claim 16 to a subject in need thereof.

18. A method for treating a condition or a disease associated with thrombosis, comprising administering the pharmaceutical composition of claim 16 to a subject in need thereof.

19. The method of claim 18, wherein the condition or the disease associated with thrombosis is myocardial infarction, pulmonary embolism, neonatal respiratory distress syndrome, adult respiratory distress syndrome, primary carcinoma of lung, non-Hodgkin's lymphoma, fibrosing alveolitis, lung transplant, atherosclerosis, surgery, stroke, or thrombosis.

20. A method for anti-coagulating blood in a subject in need thereof, comprising administering the pharmaceutical composition of claim 16 to the subject in need thereof.
